(19) United States
(12) Patent Application Publication
(10) Pub. No.: US 2009/0220558 A1
(43) Pub. Date: Sep. 3, 2009

(54) EMULSION OF A LIQUID FLUORINATED OIL PHASE IN A LIQUID WATER PHASE

(76) Inventor: David L. Malotky, Midland, MI (US)

Correspondence Address: The Dow Chemical Company, Intellectual Property Section, P.O. Box 1967, Midland, MI, 48641-1967, US

(21) Appl. No.: 12/280,412

(22) PCT Filed: Jan. 16, 2007
(86) PCT No.: PCT/US07/01124

§ 371 Date: Jan. 27, 2009

Related U.S. Application Data
(60) Provisional application No. 60/776,573, filed on Feb. 24, 2006.

(51) Int. Cl. A61K 8/06

(52) U.S. Cl. 424/401

(57) ABSTRACT

Emulsions of a liquid oil phase in a liquid water phase which comprise a) more than 50 weight percent of a hydrofluoroether, a perfluoroether or a combination thereof, based on the total weight of the emulsion, b) a surfactant, and c) water, are useful in a cosmetic formulation or a cosmetic concentrate. The hydrofluoroether is represented by formula (I) CnHmF0-O-CxHyFz wherein n is a number of 1 to 12, m is a number of 0 to 25, o is a number of 0 to 25, m+o=2n+1, x is a number of 1 to 12, y is a number of 0 to 25, z is a number of 0 to 25, and y+z=2x+1, provided that m and y are not simultaneously zero, and o and z are not simultaneously zero.

### CROSS REFERENCE STATEMENT

This application claims the benefit of U.S. Provisional Application No. 60/776,573, filed Feb. 24, 2006.

The present invention relates to an emulsion of a liquid fluorinated oil phase in a liquid water phase and to the use of such emulsion for cosmetic applications.

### BACKGROUND OF THE INVENTION

Hydrofluoroethers and perfluoroethers are a unique class of materials that can offer performance and aesthetic benefits to cosmetic products. They are also useful in the pharmaceutical field, for example as oxygen carriers, in contrast agents or in diagnostic agents. Furthermore, they are useful in cleaning compositions, such as fabric conditioners. However, because these materials are not soluble in water or cosmetic oils and have very low surface energy, they cannot be easily incorporated into cosmetic products and are very difficult to emulsify. Much research effort has been spent on the preparation of emulsions comprising hydrofluoroethers or perfluoroethers.

European Patent application EP 372 848 discloses an aqueous emulsion of a perfluoropolyether having a molecular weight of 3000 to 8000 made by agitation of the perfluoropolyether with a non-ionic non-fluorine-containing surfactant having a HLB value in the range of 11.5-17 and water. The emulsions may be used as fabric conditioners.

U.S. Pat. No. 6,113,919 discloses fluorocarbon emulsions and its use as oxygen carriers that can be employed, among other uses, as blood substitutes. European Patent No. EP 1,146,956 discloses a microemulsion which comprises a) 0.1-99 weight parts of a hydrofluoroether and b) 99.1-1 weight. parts of water, wherein the sum of a) and b) is 100 parts. The microemulsion is formed by using 5 to 30 percent of a surfactant, by the total weight of the microemulsion, wherein at least 50 percent by weight of the total amount of surfactant present in the microemulsion is a fluorinated surfactant. The European Patent No. EP 1 146 956 discloses that the microemulsion is useful as cleaning agents, solvents, and delivery systems. As discussed in this European Patent, microemulsions differ from emulsions in several ways. Microemulsions generally have particle sizes of less than 100 nm, typically form spontaneously under appropriate conditions and are thermodynamically stable. However, a dispersed phase having such small particle size requires more surfactant than an analogous emulsion in order to produce the necessary reduction in interfacial tension.

U.S. Pat. No. 5,612,043 discloses an oil-in-water emulsion for use in cosmetics and dermatology. It contains at least the following components: 0.5-50, preferably 5-20 weight percent of a perfluoropolyether, 0.5-10, preferably 1-3 weight percent of a fluoro surfactant, 0.1-0.3 weight percent of a co-emulsifying agent, 0.5 to 10 weight percent of a fatty alcohol and 10-90 weight percent of a gelled aqueous phase. The gelling agent may range from 0.1-60 weight percent of the emulsion. Vigorous stirring and heating to 80° C. is required for preparing the emulsion.

U.S. Patent Application No. 2004/0116323 discloses perfuming compositions in the form of a transparent fluid water-in-oil or oil-in-water emulsion wherein the difference between the density of the oily phase and that of the aqueous phase is less than or equal to 0.007. The dispersed phase is 5-50 percent by weight, preferably 10-35 percent by weight, of the composition. The continuous phase is 50-95 percent by weight, preferably 65-90 percent by weight of the composition. The oily phase comprises a fluorinated oil, for example a hydrofluoroether.

Although the disclosed oil-in-water emulsions provide many advantages in many applications, particularly in or as cosmetic products, it would be advantageous to find new fluorinated oil-in-water emulsions with an initially reduced amount of water, thereby providing a distinct advantage to the formulator for controlling the texture, sensation, consistency, shelf stability, and deliverability of active agents of the cosmetic product. It would be particularly advantageous to deliver the fluorinated oil into the formulation in an efficient manner to allow more space in the formulation for other ingredients and to remove the need to specially formulate a finished product to accommodate the fluorinated oil.

U.S. Patent Application No. 2004/0120975 discloses foamed oil-in-water emulsions as cosmetic carriers comprising 10-99 percent by weight of a perfluorinated compound and 1-9 percent by weight of an aqueous medium. In the perfluorinated oil-in-water dispersion the oil-based medium is physically separated from the aqueous medium. The aqueous phase is mixed with an anionic or hydrophilic emulsifier to form a base biliquid foam. A non-ionic emulsifier is used for stabilizing the foam cells after formation. The oil phase comprising a perfluorinated compound is added to the aqueous foam at an increasing rate of addition. Unfortunately, these foamed compositions have various disadvantages. For example they are inconvenient to transport and store, have limited storage stability and are limited in end-use applications. Accordingly, U.S. Patent Application No. 2004/0120975 does not satisfy the need to find new oil-in-water emulsions with an initially reduced amount of water.

Surprisingly, it has been found that emulsions of a liquid fluorinated oil phase in a liquid water phase can be produced which comprise more than 50 weight percent of a hydrofluoroether, a perfluoroether or a combination.

### SUMMARY OF THE INVENTION

One aspect of the present invention is an emulsion of a liquid oil phase in a liquid water phase in the form of a cosmetic formulation or a cosmetic concentrate which comprises a) more than 50 weight percent, based on the total weight of the emulsion, of a hydrofluoroether, a perfluoroether or a combination thereof, the hydrofluoroether being represented by formula (I)

CₙHₘFₒ—O—CₓH_{y}F_{z} (I)

wherein n is a number of 1 to 12, m is a number of 0 to 25, o is a number of 0 to 25, m+o=2n+1, x is a number of 1 to 12, y is a number of 0 to 25, z is a number of 0 to 25, and y+z=2x+1, provided that m and y are not simultaneously zero, and o and z are not simultaneously zero,

b) a surfactant, and c) water.

Another aspect of the present invention is an emulsion of a liquid oil phase in a liquid water phase which comprises

a) at least 50 weight percent, based on the total weight of the emulsion, of a hydrofluoroether of the formula (I)

CₙHₘFₒ—O—CₓH_{y}F_{z} (1)

wherein n is a number of 1 to 12, m is a number of 0 to 25, o is a number of 0 to 25, m+o=2n+1, x is a number of 1 to 12, y is a number of 0 to 25, z is a number of 0 to 25, and y+z=2x+1, provided that m and y are not simultaneously zero, and o and z are not simultaneously zero,

b) a surfactant, c) water, and d) a perfluoroether.

Yet another aspect of the present invention is a process for preparing a cosmetic formulation or a cosmetic concentrate which comprises the step of preparing an emulsion of a liquid oil phase in a liquid water phase by mixing at least

a) more than 50 weight percent, based on the total weight of the emulsion, of a hydrofluoroether of formula (I) above, a perfluoroether or a combination thereof, b) a surfactant, and c) water.

Yet another aspect of the present invention is the use of an emulsion of a liquid oil phase in a liquid water phase which comprises a) more than 50 weight percent, based on the total weight of the emulsion, of a hydrofluoroether of formula (I) above, a perfluoroether or a combination thereof, b) a surfactant, and c) water, in or as a cosmetic formulation or a cosmetic concentrate.

Yet another aspect of the present invention is process for preparing an emulsion of a liquid oil phase in a liquid water phase which comprises the step of mixing at least

a) at least 50 weight percent, based on the total weight of the emulsion, of a hydrofluoroether of formula (D) above, b) a surfactant, c) water, and d) a perfluoroether.

### DETAILED DESCRIPTION OF THE INVENTION

The emulsions of the present invention have a liquid oil phase in a liquid water phase. This means that the emulsion are substantially non-foamed. By “substantially non-foamed” is meant that the volume of a gas phase, if present in the emulsion of the present invention is not more than 20 percent, preferably not more than 10 percent, more preferably not more than 5 percent, based on the total volume of the emulsion. Most preferably no gas phase is incorporated in the emulsions.

In one aspect of the invention the emulsion of a liquid oil phase in a liquid water phase is in the form of a cosmetic formulation or a cosmetic concentrate and comprises more than 50 weight percent, preferably at least 55 weight percent, more preferably at least 60 weight percent, most preferably at least of 70 weight percent, and particularly at least 75 weight percent of a hydrofluoroether of formula (I), a perfluoroether or a combination thereof, based on the total weight of the emulsion. The upper amount of the hydrofluoroether of formula (I), the perfluoroether or a combination thereof is preferably up to 99 percent, more preferably up to 95 percent, based on the total weight of the emulsion. In those emulsions which comprise a combination of a hydrofluoroether of formula (I) and a perfluoroether, the amount of the hydrofluoroether of formula (I) is preferably from 80 to 99 percent, more preferably from 90 to 99 percent, most preferably from 95 to 99 percent, based on the total weight of the hydrofluoroether of formula (I) and the perfluoroether.

In another aspect of the present invention the emulsion of a liquid oil phase in a liquid water phase comprises at least 50 weight percent, typically more than 50 weight percent, preferably at least 55 weight percent, more preferably at least 60 weight percent, most preferably at least of 70, and particularly at least 75 weight percent of a hydrofluoroether of formula (I) and an additional amount of a perfluoroether. In such emulsion the additional amount of the perfluoroether is preferably only from 1 to 20 percent, more preferably from 1 to 10 percent, most preferably from 1 to 5 percent, based on the total weight of the hydrofluoroether of formula (I) and the perfluoroether.

The hydrofluoroether of formula (I), the perfluoroether or a combination thereof form the disperse oil phase, also called the “internal phase”. Most preferably, the emulsions of the present invention are high internal phase ratio (HIPR) oil-in-water emulsions. In general, HIPR emulsions are characterized by a disperse phase of polyhedral cells at a volume fraction of at least 74% (the most compact arrangement of spheres of equal radius) dispersed in a continuous phase that forms a thin film separating the cells.

In the hydrofluoroether of formula (I)

CₙHₘFₒ—O—CₓH_{y}F_{z} (I)

n is a number of 1 to 12, preferably of 1 to 10, more preferably of 1 to 6, and most preferably of 1 to 4; m is a number of 0 to 25, preferably of 0 to 21, more preferably of 0 to 13, most preferably of 0 to 9; o is a number of 0 to 25, preferably of 0 to 21, more preferably of 0 to 13, and most preferably of 0 to 9; m+o=2n+1, x is a number of 1 to 12, preferably of 1 to 10, more preferably of 1 to 6, and most preferably of 1 to 4; y is a number of 0 to 25, preferably of 0 to 21, more preferably of 0 to 13, and most preferably of 0 to 9; z is a number of 0 to 25, preferably of 0 to 21, more preferably of 0 to 13, and most preferably of 0 to 9; and y+z=2x+1, provided that m and y are not simultaneously zero, and o and z are not simultaneously zero.

Preferably, the hydrofluoroether is of the formula I′

CₙF₂ₙ₊₁—O—CₓH₂ₓ₊₁ (I′),

wherein n is a number of 1 to 12, preferably of 1 to 10, more preferably of 1 to 6, most preferably of 1 to 4, and x is a number of 1 to 12, preferably of 110, more preferably of 1 to 6, most preferably of 1 to 4.

Specific examples of the hydrofluoroether include C₄F₉—O—CH₃, C₄F₉—O—C₂H₅, C₄F₉—O—C₃H₇, C₅F₁₁—O—C₂H₅, C₃F₇—O—C₄H₉ and C₄F₉—O—C₄H₉ The most preferred hydrofluoroether is C₄F₉—O—C₂H₅.

The emulsion of the present invention can comprise one, two or more kinds of hydrofluoroethers of the formula I, however the total amount of the hydrofluoroether(s) of the formula I should be within the ranges indicated herein.

If the emulsion of the present invention mainly comprises a hydrofluoroether of formula (I) of a low boiling point, that means a boiling point of generally less than 100° C. at ambient pressure and temperature, typically of less than 80° C. at ambient pressure and temperature, it is generally desirable to also include a hydrofluoroether of formula (I) of a boiling point of at least 110° C., preferably a boiling point of a least 125° C., or a perfluoroether to enhance the stability of the emulsion. The amount of the hydrofluoroether of formula (I) of higher boiling point or the perfluoroether preferably is from 1 to 20 percent, more preferably from 1 to 10 percent, most preferably from 1 to 5 percent, based on the total weight of all hydrofluoroethers of formula (I) and optionally the perfluoroether.

The term “perfluoroether” as used herein means a fully fluorinated monomeric or polymeric compound essentially consisting of carbon atoms, fluorine atoms and oxygen atoms.

Preferably, the perfluoroether is a perfluoropolyether which is liquid at room temperature and atmospheric pressure. The perfluoropolyethers utilizable in the emulsions of the invention are compounds which comprise perfluoroalkylene oxide units or perfluoroxetane rings. In particular, the perfluoropolyether contains repeating units chosen from the following:

(A) C₂F₄O and CF₂O statistically distributed along the chain;

(B) C₂F₄O, C₃F₆O and CFZO, wherein Z is F or CF₃, statistically distributed along the chain;

(C)C₃F₆O and CFZO, wherein Z is F or CF₃, statistically distributed along the chain; or

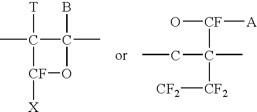

(D) oxetane rings

in which A, T and X are equal to or different from each other and are perfluorooxyalkyl, perfluoropolyoxyalkyl or perfluoroalkyl radicals.

The end groups of the perfluoropolyethers may be like or unlike each other and are preferably selected from the radicals F, CF₃, C₂F₅ and C₃F₇. The mean number average molecular weight is generally higher than 500 and ranges in particular from 1,000 to 10,000. The viscosity values are generally in the range of from 30 to 5,000 cSt (corresponding to 30×10⁻⁶ to 5,000×10⁻⁶ m²/s) at 20° C.

Specific examples of perfluoropolyethers are:

(i) CF₃O—(C₃F₆O)ₚ—(CFZ¹O)_{q}—CF₂Z², wherein Z¹ and Z² each independently are F or CF₃, and p and q are integers, and the p/q ratio ranges from 5 to 40; these compounds and the method of preparing them are described in British Pat. No. 1,104,482.

(ii) C₃F₇O—(C₃F₆O)ᵣ—R_{f}, wherein R_{f} is C₂F₅ or C₃F₇, and r is an integer higher than 2, preferably from 10 to 100; these compounds and the method of preparing them are described in the U.S. Pat. No. 3,242,218; examples of such compounds are sold under the trademark DEMNUM manufactured by Daikin Industries, Ltd.).

(iii) CF₃O—(C₃F₆O)ₚ—(C₂F₄O)ₛ—(CFZO)_{q}—CF₃, wherein Z is F or CF₃, p, q and s are integers, the sum of p+s+q is from 10 to 300, the s/q ratio ranges from 0.5 to 5, and the p/(s+q) ratio ranges from 0.0 to 0.4; these compounds and the method of preparing them are described in U.S. Pat. No. 3,665,041;

(iv) CF₃O—(C₂F₄O)ₛ—(CF₂O)ₜ—CF₃, wherein s and t are integers alike or different from each other and the s/t-ratio ranges from 0.5 to 1.5; examples of these compounds and the method of preparing them are described in U.S. Pat. Nos. 3,715,378 and 3,665,041; examples of such compounds are sold und the trademark FOMBLIN Z by Montefluos Ltd., Montedison Group, Italy;

(v) F—(CF(CF₃)—CF₂O)ᵤ—CF₂—CF₃, wherein u is an integer from 4 to 500; examples of such compounds are sold und the trademark KRYTOX by Du Pont de Nemours;

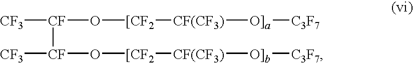

wherein a and b independently are integers from 0 to 3;

vii) compounds having the oxetane-structure as described in Italian Patent Application No. 19496 A/85; and

(viii) perfluoropolyethers comprising CF₂CF₂CF₂O units and perfluoropolyethers comprising CF₂CF₂O units; such perfluoropolyethers and their preparation are described in EP published application No. 148,482 and U.S. Pat. No. 4,523,039.

In the formulas above p, q, r, s, t and u are preferably chosen that the weight average molecular weight is higher than 500 and preferably from 1,000 to 10,000.

The preferred liquid perfluoropolyethers in the emulsions of the present invention are perfluoropolymethylisopropyl ethers which are known under the IUPAC name trifluoromethyl-poly[oxy-2-trifluoromethyl-1,1,2-trifluoroethylene]-poly [oxy-difluoromethylene]-trifluoromethyl ether. The preferred perfluoropolymethylisopropyl ethers have the following chemical structures:

CF₃—[O—CF(CF₃)—CF₂]ᵥ—(O—CF₂)_{w}—O—CF₃, wherein v and w are integers and the v/w ratio is from 5 to 40 and v and w are chosen that the weight average molecular weight is higher than 500 and preferably from 1,000 to 10,000.

These compounds are generally manufactured by Montefluos S.p.A and sold under the names Fomblin HC and Fomblin Y. Such compounds are disclosed in U.S. Pat. No. 4,803,067, U.S. Pat. No. 4,959,171 and U.S. Pat. No. 5,093,023, which are incorporated herein by reference in their entirety. Exemplary of such Fomblin compounds include Fomblin HC products, such as Fomblin HC-04 with a weight average molecular weight of 1500, Fomblin HC-25 with a weight average molecular weight of 3200 and Fomblin HCR with a weight average molecular weight of 6250; Fomblin Y products, such as Fomblin Y04 with a weight average molecular weight of 1,500, Fomblin Y25 with a weight average molecular weight of 3,000 and Fomblin YR with a weight average molecular weight of 6,000-7,000.

The emulsion of the present invention can comprise one, two or more kinds of perfluoroethers.

The emulsion of the present invention in the form of a cosmetic formulation or a cosmetic concentrate can comprise i) one, two or more kinds of hydrofluoroethers of the formula I, or ii) one, two or more kinds of perfluoroethers or iii) a combination of one, two or more kinds of hydrofluoroethers of the formula I and one, two or more kinds of perfluoroethers, provided that their total amount is more than 50 weight percent, based on the total weight of the emulsion.

The emulsions of the present invention also comprise a surfactant b) for stabilizing the emulsion. The amount of the surfactant preferably is from 0.5 percent, more preferably from 1 percent, most preferably from 2 percent, and preferably up to 20 percent, more preferably up to 10 percent, and most preferably up to 5 percent, based on the total weight of the emulsion. Useful surfactants include nonionic, anionic, or cationic surfactants, or combinations of nonionic and anionic or nonionic and cationic surfactants.

Suitable nonionic surfactants are disclosed in U.S. Pat. No. 3,929,678, Laughlin et al., and U.S. Pat. No. 4,285,841, Barrat et al. Examples of nonionic surfactants suitable for stabilizing the emulsion include polyethylene glycol fatty acid mono- and diesters (such as PEG-8 laurate, PEG-10 oleate, PEG-8 dioleate, and PEG-12 distearate), polyethylene glycol glycerol fatty acid esters (such as PEG-40 glyceryl laurate and PEG-20 glyceryl stearate), alcohol-oil transesterification products (such as PEG-35 castor oil, PEG-25 trioleate, and PEG-60 corn glycerides), polyglycerized fatty acids (such as polyglyceryl-2-oleate and polyglyceryl-10 trioleate), propylene glycol fatty acid esters (such as propylene glycol monolaurate), mono- and diglycerides (such as glyceryl monooleate and glyceryl laurate), sterol and sterol derivatives (such as cholesterol), sorbitan fatty acid esters and polyethylene glycol sorbitan fatty acid esters (such as sorbitan monolaurate and PEG-20 sorbitan monolaurate), polyethylene glycol alkyl ethers (such as PEG-3 oleyl ether and PEG-20 stearyl ether), sugar esters (such as sucrose monopalmitate and sucrose monolaurate), polyethylene glycol alkyl phenols (such as PEG-10-100 nonyl phenol, and PEG-15-100 octyl phenol ether), polyoxyethylene-polyoxypropylene block copolymers (such as poloxamer 108 and poloxamer 182), and lower alcohol fatty acid esters (such as ethyl oleate and isopropyl myristate).

Examples of suitable ionic surfactants include fatty acid salts (such as sodium laurate and sodium lauryl scarcosinate), bile salts (such as sodium cholate and sodium taurocholate), phospholipids (such as egg/soy lecithin and hydroxylecithin), phosphoric acid esters (such as diethanolammonium polyoxyethylene-10 oleyl ether phosphate), carboxylates (such as ether carboxylates and citric acid esters of mono and diglycerides), acyl lactylates (such as lactylic esters of fatty acids, and propylene glycol aginate), sulfates and sulfonates (such as ethoxylated alkyl sulfates, alkyl benzene sulfones, and acyl taurates), and alkyl, aryl, and alkyl-aryl sulfonates and phosphates. Examples of suitable cationic surfactants include quaternary ammonium salts and hydrochloride salts of N-alkyl diamines and triamines.

Preferred nonionic surfactants include C₈-C₁₈ alkyl ethoxylates, with about 1-22 ethylene oxide units, including the so-called narrow peaked alkyl ethoxylates and C₆-C₁₂ alkyl phenol alkoxylates, particularly ethoxylates and mixed ethoxylates/propoxylates, alkyl dialkyl amine oxides, alkanoyl glucose amides, and mixtures thereof. Other useful nonionic surfactants are polyethylene, polypropylene, and polybutylene oxide condensates of alkyl phenols. Commercially available nonionic surfactants of this type include Igepal® CO-630, marketed by the GAF Corporation; and Triton® X45, X-114, X-100, and X-102, all marketed by the Rohm & Haas Company. These compounds are commonly referred to as alkyl phenol alkoxylates, preferably alkyl phenol ethoxylates. Further useful nonionic surfactants are the condensation products of aliphatic alcohols with from 1 to 25 moles of ethylene oxide. The alkyl chain of the aliphatic alcohol can either be straight or branched, primary or secondary, and generally contains from 8 to 22 carbon atoms. Examples of commercially available nonionic surfactants of this type include Tergitol® 15-S-9 (the condensation product of C₁₁-C₁₅ linear secondary alcohol with 9 moles ethylene oxide), Tergitol® 24-L-6 NMW (the condensation product of C₁₂-C₁₄ primary alcohol with 6 moles ethylene oxide with a narrow molecular weight distribution), both marketed by The Dow Chemical Corporation; Neodol® 45-9 (the condensation product of C₁₄-C₁₅ linear alcohol with 9 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, and Kyro® EOB (the condensation product of C₁₃-C₁₅ alcohol with 9 moles ethylene oxide), marketed by The Procter & Gamble Company. Other commercially available nonionic surfactants include Dobanol 91-8(marketed by Shell Chemical Co. and Genapol UD-080® marketed by Hoechst. This category of nonionic surfactant is referred to generally as “alkyl ethoxylates.” Other useful nonionic surfactants are the condensation products of ethylene oxide with a hydrophobic base formed by the condensation of propylene oxide with propylene glycol. Examples of compounds of this type include certain of the commercially-available Pluronic® surfactants, marketed by BASF. Further useful surfactants are the condensation products of ethylene oxide with the product resulting from the reaction of propylene oxide and ethylenediamine. Examples of this type of nonionic surfactants include certain of the commercially available Tetronic® compounds, marketed by BASF. Semi-polar nonionic surfactants are a special category of nonionic surfactants which include water-soluble amine oxides. These amine oxide surfactants in particular include C₁₀-C₁₈ alkyl dimethyl amine oxides and C₈-C₁₂ alkoxy ethyl dihydroxy ethyl amine oxides. Other nonionic surfactants are alkylpolysaccharides. Any reducing saccharide containing 5 or 6 carbon atoms can be used, for example, glucose, galactose and galactosyl moieties can be substituted for the glucosyl moieties. Fatty acid amide surfactants, C₁₂-C₁₈ betaines and sulfobetaines (sultaines) are also knows surfactants.

The most preferred nonionic surfactants include linear alkyl ethoxylates, such as the Brij series from Uniqema, which are polyethylene oxide fatty ethers derived from lauryl, stearyl, cetyl or oleyl alcohols. Polyethylene oxide fatty ethers derived from lauryl alcohol, or cetyl alcohol, particularly polyethyleneoxide (4) lauryl ether, which is commercially available under the trademark Brij 30; polyethyleneoxide (23) lauryl ether, which is commercially available under the trademark Brij 35; and polyethyleneoxide (20) cetyl ether, which is commercially available under the trademark Brij 58; are particularly preferred. Other preferred non-ionic surfactants are secondary alcohol ethoxylates such as the Tergitol™ 15-S series from The Dow Chemical Company.

Preferably, the emulsions may contain an additive to prevent Ostwald ripening, that is, to prevent diffusion of the oil disperse phase from small droplets of the disperse phase to larger ones. The additive used to prevent Ostwald ripening is a highly water-insoluble material that 1) has a negligible diffusion coefficient in the continuous aqueous phase and 2) is compatible with the disperse phase, for example, a natural oil such as cottonseed oil or sunflower seed oil, an oil phase compatible polymer such as polyisobutene, or polyethylene; a long chain paraffin such as hexadecane; or a silicone such as silicone oil or dimethicone. Preferably, the additive used to prevent Ostwald ripening is used in an amount not greater than 5 weight percent, and more preferably not greater than 2 weight percent, based on the weight of the oil disperse phase and the additive. In those embodiments of the present invention which comprise at least 50 weight percent of a hydrofluoroether of formula (I), an above-described perfluoroether or hydrofluoroether with boiling point at least 110° C. can be used as an additive to prevent Ostwald ripening.

Optionally the emulsions of the present invention include non-fluorinated diluents that are miscible with the hydrofluoroether of formula (I) or the perfluoroether in the oil phase of the emulsion. Examples of non-fluorinated materials are alcohols, such as ethanol, 1-propanol, 1-butanol, 1-pentanol, 1-hexanol, 1-heptanol, or 1-octanol, alkanes, such as C₅ to C₁₂-alkanes, isododecane, Isopar H, or Isopar M, glycol ethers or glycol esters, such as isopropyl myristate and isopropyl palmitate, or silicones, such as cyclomethicone, disiloxane, trisiloxane, Dow Corning OS-10, or Dow Corning OS-20. The emulsions of the present invention may also comprise water-insoluble materials which are not miscible or only partially miscible with the hydrofluoroether of formula (I) or the perfluoroether, such as ketones like acetone, chlorinated solvents, aromatic solvents, mineral oil, dimethicone, cetearyl isononanoate, octyl dodecanoate, and caprylic/capric triglyceride. The optional additional diluents are generally present in the emulsion of the present invention from 0 percent to 40 percent, preferably from 0 percent to 30 percent, and more preferably from 0 to 10 percent, based on the total weight of the emulsion.

Other ingredients that may optionally be present in the emulsion of the present invention may include adjuvants or benefit agents, for example fragrances, antioxidants, chelating agents, UV filters, preservatives, thickening agents, cosmetic active ingredients, moisturizers, humectants, emollients, opacifiers, pearly gloss impacting substances, pigments, colorants, dyes or antifoaming agents. The optional additional ingredients are generally present in the composition of the present invention from 0 to 5 percent, preferably from 0.5 to 3 percent, more preferably from 0.1 to 1 percent, based on the total weight of the emulsion.

The emulsions of the present invention also comprises water. Its amount is dependent on the weight percentages of the other ingredients on the emulsion. It is advantageous to add a preservative to the water.

The emulsion of the present invention generally has a volume mean particle size of more than 0.2 microns (μm) and typically more than 0.5 microns. The emulsion of the present invention generally has a volume mean particle size of up to 50 microns (μm), preferably up to 20 microns, more preferably up to 10 microns, and most preferably up to 5 microns, measured using a Coulter LS 230 Laser Light Scattering Particle Sizer (Coulter Corp.).

The emulsion can be prepared by a variety of methods, preferably mechanical dispersions techniques including batch and continuous methods well-known in the art. In a preferred continuous method (described generally by Pate et al. in U.S. Pat. No. 5,539,021, column 3, line 15 to column 6, line 27, which passage is incorporated herein by reference), a first stream containing a continuous aqueous phase is flowed through a first conduit and merged continuously with a second stream of a dispersed oil phase that is flowed through a second conduit. The first and second streams are merged into a disperser in the presence of a stabilizing amount of surfactant. The surfactant can be added to either the first or second stream, or as a separate third stream, but is preferably added to the stream containing the oil phase. The rates of the streams are adjusted within the emulsion region to achieve an emulsion having the above-mentioned percentage of oil phase comprising a hydrofluoroether of formula (I), a perfluoroether or a combination thereof. The weight rations between the streams of oil phase: water phase: surfactant in a continuous process generally is 5-10:1-15:1, preferably 10-40:1-4:1,more preferably 10-30:2:1, most preferably 15-25:2:1. If the preferred method used for preparing the emulsion is a continuous method, the oil phase must be flowable through a conduit.

If a hydrofluoroether of formula (I), a perfluoroether or a combination thereof is used which is sufficiently low in viscosity to be flowable at ambient temperature and without dilution with a solvent, the emulsion is preferably prepared at ambient temperature and without the use of a solvent. If, on the other hand, a hydrofluoroether of formula (I), a perfluoroether or a combination thereof is used which is not flowable through a conduit at ambient temperature, either because it is a solid or a highly viscous liquid at ambient temperature, it can be rendered flowable by either heat or solvent addition. Minor amounts, such as, for example, not greater than 5 percent, preferably not greater than 1 percent, and more preferably not greater than 0.5 percent by weight, of water-compatible substances, that is, substances which, by themselves are incapable of forming oil-in-water emulsions, can be added to the oil phase prior to emulsification of the oil phase. Examples of such water-compatible substances include rheology modifiers such as carbomers, acrylic copolymers, polyacrylamides, polysaccharides, natural gums, and clays; preservatives such as alkyl esters of p-hydroxybenzoic acid; humectants such as glycerol; pH modifiers; and mixtures thereof.

Surprising, it has also been found that the emulsions of the present invention can be produced in a batch process. In a batch process the ratio oil phase:water phase:surfactant preferably is from generally is 3-40:2-8:1, preferably 4-30:3-6:1, more preferably 5-20:4-5:1. Preferably the surfactant and water are combined in an above-mentioned ratio. Heating may be in some cases advisable to melt the surfactant and to create a uniform aqueous phase. Preferably the aqueous phase is allowed to cool to room temperature after the optional heating step and subjected to shear while the hydrofluoroether of formula (I), a perfluoroether or a combination thereof is added. However, an above-described continuous method is generally preferred because an emulsion of smaller particle size and higher stability is usually obtained than in a batch process.

The emulsions of the present invention are surprisingly stable. In general they have found to be stable for at least 4 weeks at a temperature of 50° C. or at least 12 weeks at a temperature of 45° C. without an appreciable increase in volume mean particle size. In general, the emulsions of the present invention are also stable to multiple freeze thaw cycles. The emulsion of the present may be combined with a partial cosmetic formulation at any volume ratios to produce an advanced cosmetic product. As used herein, “partial cosmetic formulation” refers to one or more finishing ingredients, which, when combined with the emulsion of the present invention, form an advanced, and preferably a finished, cosmetic product. The term “advanced cosmetic product” refers to either a finished cosmetic product or one that is closer to being a finished product than before the emulsion of the present invention and partial cosmetic formulation were combined. Preferably, the advanced cosmetic product is a finished cosmetic product that is ready to be packaged for and sold to the consumer. In an extreme case, the emulsion of the present invention may contain all of the ingredients of the finished product, and the partial cosmetic formulation is simply water. In this case, the emulsion of the present invention represents a concentrate of the finished product, which is merely diluted with water to form the finished product. Simple blending at room temperature is generally sufficient to prepare an advanced cosmetic product. Usually no specialized processing technique or blending devices are necessary.

According to the present invention it is possible to prepare an emulsion that includes ingredients commonly found in a partial cosmetic formulation such as color, fragrance, rheology modifier, pH adjuster, and other ingredients such as active agents, aesthetics modifying agents, and adjuvants as described in WO 01/54663, page 15 to page 22, incorporated herein by reference. However, it may be desirable to exclude the above ingredients from the emulsion of the present invention. For example, color, fragrance, rheology, or pH may be more easily controlled when included in the partial cosmetic formulation and combined with an emulsion of the present invention that contains predominantly the emulsified hydrofluoroether of formula (I), a perfluoroether or a combination thereof. The emulsion of the present invention and the partial cosmetic formulation can be combined concomitantly or in any order. Furthermore, more than one emulsion can be combined with the partial cosmetic formulation to form the advanced cosmetic product. For example, one or more emulsions of the present invention can be combined with a partial cosmetic formulation containing water, thickener, fragrance, and color to form a body lotion. Examples of finished cosmetic products include hand lotions, body lotions, body washes, conditioners, shampoos, facial creams, facial lotions, facial masks, and sunscreens compositions. Due to the incorporation of a hydrofluoroether of formula (I), a perfluoroether or a combination thereof in the cosmetics products, the cosmetic products have superior protective and spreading properties.

The process of the present invention provides a simple and flexible method of formulating the cosmetic product, due to the ease with which an emulsion with controlled particle size can be reproduced, due to the long shelf-stability of the emulsion, and due to the low quantity of water in the emulsion.

The emulsion of the present invention which comprises a) at least 50 weight percent of a hydrofluoroether of formula (I), b) a surfactant, c) water, and d) a perfluoroether is particularly, but not exclusively useful in or as a cosmetic composition or cosmetic concentrate. Other uses are in therapeutic or diagnostic compositions for administering bioactive agents. Examples of pharmaceutical uses are oxygen carriers that can be employed, among other uses, as blood substitutes or as concentrates for producing blood substitutes. Examples of diagnostic uses are contrast agents to facilitate diagnosis, particularly by radiography, sonography or nuclear magnetic resonance imagery. The emulsions are also useful in cleaning agents or fabric conditioning compositions. The emulsions are also useful as heat transfer fluids, as lubricants or as metal working fluids. The emulsions are also useful as coatings to modify the surface properties of materials such as paper, fabric, or masonry. However, the use of the emulsions in or as cosmetic compositions or cosmetic concentrates is particularly preferred.

The following examples are for illustrative purposes only and are not intended to limit the scope of the present invention. All percentages are by weight unless otherwise specified. Particle sizes of the emulsions are measured using a Beckman Coulter LS 230 Particle Size Analyzer.

### Example 1

### Continuous Method

An oil phase stream of 98 percent CF-76 Cosmetic Fluid from 3M (ethyl perfluorobutyl ether, ethyl perfluoroisobutyl ether, density=1.43 g/ml) and 2 percent Fomblin HC-25 (Trademark) is supplied at room temperature to a 2″ (2 inch, corresponding to 5.08 cm) Oakes rotor stator mixer at 10 ml/min. Fomblin HC-25 is a trademark for perfluoropolymethylisopropyl ether with a density=1.9 g/ml and a weight average molecular weight of 3200. Also supplied to the mixer are i) an aqueous stream at 1.2 ml/min, that has room temperature and contains water and a preservative package of 0-6 percent phenoxyethanol, 0.2 percent EDTA, and 0.2 percent potassium sorbate, based on the total weight of the aqueous stream, and ii) a surfactant stream at 0.6 ml/min. that is a mixture of 50 percent Brij 30 (trademark for polyethyleneoxide (4) lauryl ether) and 50 percent Brij 35 (trademark for polyethyleneoxide (23) lauryl ether). The surfactant stream is supplied at a temperature of 50° C. and is merged with the water stream before it enters the mixer. The mixer is operated at 1000 rpm with a jacket temperature of 15° C. The emulsion created at these conditions comprises 87.9 percent disperse oil phase, based on the total weight of the composition, and has a volume mean particle size of 1.1 microns (em).

### Example 2

### Continuous Method

An oil phase stream of 100 percent Fomblin HC-25 (Trademark for perfluoropolymethylisopropyl ether) is supplied at room temperature to the same 2″ Oakes rotor stator mixer as in Example 1 at 20 ml/min. The same aqueous stream as in Example 1 at 1.8 ml/min. and the same surfactant stream as in Example 1 at 1.2 g/min. are also supplied to the mixer. The water stream and the surfactant stream are merged as in Example 1 and the mixer is operated as in Example 1. The emulsion created at these conditions comprises 92.7 percent disperse oil phase and has a volume mean particle size of 3.2 microns.

### Example 3

### Batch Process

9.3 g of water, 1.5 g of Brij 35 (trademark for polyethyleneoxide (23) lauryl ether) and 0.6 g of Brij 58 (trademark for polyethyleneoxide (20) cetyl ether) are combined in a 2 oz (60 ml) glass jar, which is heated to melt the surfactants and mixed to form a uniform solution. Once the surfactant solution is homogeneous, it is cooled to room temperature and agitated with a Cowles blade mixer at 500 rpm. As the surfactant solution is agitated, an oil phase of CF-76 Cosmetic Fluid is slowly added until 40.1 g has been incorporated into an emulsion. This emulsion comprises 78 percent disperse oil phase and has a volume mean particle size of 21.7 (Jim) microns. A clear layer of water begins to form on the surface of this emulsion after two days at room temperature, although the particle size of the emulsion does not change considerably. The volume mean particle size is 18.9 microns after 2 days.

### Example 4

### Batch Process

15 g of water, 2.5 g of Brij 30 (trademark for polyethyleneoxide (4) lauryl ether) and 2.5 g of Brij 35 (trademark for polyethyleneoxide (23) lauryl ether) are combined in a 2 oz (60 ml) glass jar, which is heated to melt the surfactants and mixed to form a uniform solution. Once the surfactant solution is homogeneous, it is cooled to room temperature and agitated with an IKA Ultra Turex mixer at 11,000 rpm. As the surfactant solution is agitated, an oil phase as in Example 1 consisting of 98 percent CF-76 Cosmetic Fluid and 2 percent Fomblin HC-25 is slowly added until 49.25 g has been incorporated into the emulsion. This emulsion comprises 71.1 percent disperse oil phase and has a volume mean particle size of 1.82 microns.

### Example 5

### Batch Process

10.5 g of water and 3.57 g of Tergitol 15-S-12 (Trademark of The Dow Chemical Company for C11-15 pareth-12 (alkyloxypolyethyleneoxyethanol) are combined, heated, cooled and agitated as in Example 4. The same oil phase as in Example 1 consisting of 98 percent CF-76 Cosmetic Fluid and 2 percent Fomblin HC-25 is slowly added until 42.38 g has been incorporated into the emulsion. This emulsion comprises 75.1 percent oil phase and has a volume mean particle size of 0.99 microns.

### Example 6

### Batch Process

16.65 g of water and 5.57 g of Tergitol 15-S-12 are combined, heated, cooled and agitated as in Example 5. An oil phase consisting of 100% Fomblin HC-25 (perfluoropolymethylisopropyl ether) is slowly added until 23.22 g has been incorporated into an emulsion. This emulsion comprises 51.2 percent oil phase and has a volume mean particle size of 10.23 microns.

1. A cosmetic formulation or concentrate comprising:
an emulsion of a liquid oil phase in a liquid water phase comprising
a) more than 50 weight percent, based on the total weight of the emulsion, of a hydrofluoroether, a perfluoroether or a combination thereof, the hydrofluoroether being represented by formula (I)
CₙHₘFₒ—O—CₓH_{y}F_{z} (I)
wherein n is a number of 1 to 12, m is a number of 0 to 25, o is a number of 0 to 25, m+o=2n+1, x is a number of 1 to 12, y is a number of 0 to 25, z is a number of 0 to 25, and y+z=2x+1, provided that m and y are not simultaneously zero, and o and z are not simultaneously zero,
b) a surfactant, and
c) water.

2. The cosmetic of claim 1, wherein the emulsion comprises at least 60 weight percent of a hydrofluoroether of formula (I), a perfluoroether or a combination thereof.

3. The cosmetic of claim 1, further comprising
at least 50 weight percent, based on the total weight of the emulsion, of a hydrofluoroether of the formula (I)
d) a perfluoroether.

4. The cosmetic of claim 3 comprising at least 60 weight percent of a hydrofluoroether of formula (I).

5. The cosmetic of claim 1 wherein the perfluoroether is a perfluoropolyether essentially consisting of carbon atoms, fluorine atoms and oxygen atoms and comprising perfluoroalkylene oxide units or perfluoroxetane rings.

6. The cosmetic of claim 1 wherein the emulsion has a volume mean particle size of up to 50 microns.

7. The cosmetic claim 1 additionally comprising up to 20 weight percent of a polyhydroxylated compound having three or more hydroxyl groups, based on the total weight of the emulsion.

8.-10. (canceled)

11. The cosmetic of claim 1, wherein the emulsion is substantially non-foamed.

12. The cosmetic of claim 1, wherein the emulsion has a hydrofluoroether of formula (I) with boiling point of less than 100° C. at ambient pressure and temperature combined with at least one of a hydrofluoroether of formula (I) with a boiling point of at least 110° C. or a perfluoroether.

13. The cosmetic of claim 12, wherein the amount of the hydrofluoroether of formula (I) of higher boiling point or the perfluoroether preferably is from 1 to 20 percent based on the total weight of all hydrofluoroethers of formula (I) and the perfluoroether when present.

14. The cosmetic of claim 1, wherein the hydrofluoroether is C₄F₉—O—C₂H₅.

15. The cosmetic of claim 1, wherein the perfluoroether is a perfluoropolyether.

16. The cosmetic of claim 15, wherein the perfluoropolyether is trifluoromethyl-poly[oxy-2-trifluoromethyl-1,1,2-trifluoroethylene]-poly[oxy-difluoromethylene]-trifluoromethyl ether.

17. The cosmetic of claim 1, further comprising at least one fragrance, antioxidant, chelating agent, UV filter, preservative, thickening agent, cosmetic active ingredient, moisturizer, humectant, emollient, opacifier, pearly gloss impacting substance, pigment, colorant, dye, or antifoaming agent.

18. The cosmetic of claim 1, wherein the emulsion is produced in a continuous process, where the weight ratio between oil phase:water phase:surfactant is 10-30:2:1.

19. The cosmetic of claim 1, wherein the emulsion is produced in a batch process, where the weight ratio between oil phase:water phase:surfactant is 4-30:3-6:1.
